(19) United States
(12) Patent Application Publication
(10) Pub. No.: US 2002/0041907 A1
(43) Pub. Date: Apr. 11, 2002

(54) Sophora japonica extracts for promoting desquamation/epidermal renewal of the skin and/or combating skin aging

(76) Inventors: Christel Liviero, Paris (FR); Pascale Pelletier, Antony (FR); Lionel Breton, Versailles (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/906,734

(22) Filed: Jul. 18, 2001

(30) Foreign Application Priority Data
Jul. 18, 2000 (FR) 00/09403

(51) Int. Cl. A61K 35/78

(52) U.S. Cl. 424/725

(57) ABSTRACT

A regime or regimen for promoting desquamation and/or for stimulating epidermal renewal and/or for combating intrinsic/extrinsic aging of the skin of individuals in need of such treatment, comprises administering to such individuals, whether orally, injectably or topically, for such period of time as required to elicit the desired response, a thus effective amount of at least one extract of at least one plant of the species Sophora japonica.

### CROSS-REFERENCE TO PRIORITY APPLICATION

This application claims priority under 35 U.S.C. §119 of FR-00/09403, filed Jul. 18, 2000, hereby expressly incorporated by reference.

### BACKGROUND OF THE INVENTION

1. Technical Field of the Invention

The present invention relates to the administration of at least one extract of at least one plant of the species Sophora japonica, or composition comprised thereof, to promote desquamation of the skin and/or to stimulate epidermal renewal and/or to combat skin aging.

The present invention also relates to compositions for promoting desquamation of the skin and/or stimulating epidermal renewal and therefore for combating intrinsic and/or extrinsic skin aging, as well as to a nontherapeutic regime/regimen of treating the skin to promote desquamation and/or combat skin aging.

2. Description of the Prior Art

Desquamation is a natural phenomenon linked to the fact that the epidermis, which constitutes the top or outer layer of the skin, is constantly being regenerated. The epidermis consists of several layers of cells, of which the deepest is the basal layer consisting of undifferentiated cells. Over time, these cells will differentiate and migrate to the surface of the epidermis, constituting various layers thereof, until the corneocytes, which are dead cells which are eliminated by desquamation, are formed at the surface of the epidermis. This loss at the surface is compensated by the migration of cells from the basal layer to the surface of the epidermis. It entails the perpetual renewal of the skin. The forced elimination of the horny layer accelerates epidermal renewal and makes it possible to combat aging.

At the same time, these cells continue their differentiation, of which the last stage is the corneocyte. They are dead cells which constitute the last layer of the epidermis, namely, the outermost layer also termed stratum corneum.

Skin aging, resulting from effects of intrinsic or extrinsic factors on the skin, results in the appearance of wrinkles and fine lines, in yellowing of the skin, which develops in particular a shrivelled appearance, accompanied by the appearance of pigmentation marks, in the disorganization of the elastin fibers and of collagen, causing a loss of elasticity, suppleness and firmness and in the appearance of telangiectasia.

Certain of these signs of aging are more particularly associated with intrinsic or physiological aging, namely, to “normal” age-related or chronobiological aging, whereas others are more specific to extrinsic aging, namely, aging generally caused by the environment; this is, more particularly, photoaging due to exposure to the sun, to light or to any other radiation.

The present invention relates to intrinsic or physiological aging as well as to extrinsic aging.

Changes in the skin due to intrinsic aging are the consequence of a genetically programmed aging in which endogenous factors are involved. This intrinsic aging causes, in particular, a slowing down of the renewal of the skin cells, which essentially results in the appearance of clinical alterations such as the reduction in the subcutaneous adipose tissue and the appearance of fine wrinkles or fine lines, and in histopathological changes, such as an increase in the number and thickness of the elastic fibers, a loss of vertical fibers of the membrane of the elastic tissue, and the presence of large irregular fibroblasts in the cells of this elastic tissue.

In contrast, extrinsic aging causes clinical alterations such as thick wrinkles and the formation of a soft and tanned skin, and histopathological changes such as an excessive accumulation of elastic material in the upper dermis and degeneration of the collagen fibers.

Various active agents for combating skin aging are known to this art.

Thus, U.S. Pat. No. 4,603,146 describes the formulation of retinoic acid and derivatives thereof into cosmetic compositions, for combating skin aging.

Moreover, numerous patents and publications (see, for example, EP-A-0,413,528) as well as numerous commercially available cosmetic compositions suggest administering, or contain, α-hydroxy acids such as lactic acid, glycolic acid or, alternatively, citric acid for treating skin aging.

Too, the β-hydroxy acids and, more especially, salicylic acid, as well as derivatives thereof, are known for their desquamative properties (see WO-A-93/10756 and U.S. Pat. No. 4,767,750).

All of the above compounds exhibit activity against skin aging by promoting desquamation, i.e., the elimination of the “dead” cells situated at the surface of the horny layer of the epidermis. This “desquamative” property is also designated, often wrongly, keratolytic property.

However, the prior art compounds also have adverse side effects such as prickling, twitching, overheating and redness which are unpleasant for the user.

Serious need, therefore, continues to exist for anti-aging agents eliciting an action which is at least as effective as that of the prior art compounds, but which do not present the disadvantages/drawbacks thereof.

### SUMMARY OF THE INVENTION

Accordingly, a major object of the present invention is the provision of unique regime or regimen for promoting desquamation of the skin and/or stimulating epidermal renewal, while at the same time avoiding or conspicuously ameliorating the sensations of prickling, twitching, overheating or redness which are unpleasant for the user.

Briefly, it has now unexpectedly and surprisingly been found that administration of at least one extract of at least one plant of the species Sophora japonica promotes desquamation of human skin and/or stimulates epidermal renewal and, therefore, combats skin aging.

### DETAILED DESCRIPTION OF BEST MODE AND SPECIFIC/PREFERRED EMBODIMENTS OF THE INVENTION

More particularly according to the present invention, in the prior art publications, extracts of at least one plant of the species Sophora japonica are described for their dyeing property, particularly in dyeing silk, but also in pharmacology because of their rutoside content (Jean Bruneton, Phytochimie, Plantes Médicinales (Phytochemistry, Medicinal Plants) 2nd edition, techniques et documentation-lavoisier, p. 281, 1993).

Antibacterial properties have also been described (Kimura M, Yamada H; “Interaction in the antibacterial activity of flavonoids from sophra japonica L. to Propionibacterium”; Yakugaku Zasshi; 104 (4); 340-6; 1984).

Antihemorrhagic properties are also indicated in the prior art (Ishida H, Umino T.: “Studies on Antihemorrhagic Substances in Herbs Classified as Hemo statics in Chinese Medicine. VI. On the Antihemorrhagic principle in Sophora japonica L.”, Chem Pharm Bull; 35(2): 857-60; 1987).

To date, however, as best as can be determined, it was unknown to use at least one extract of at least one plant of the species Sophora japonica for promoting desquamation of the skin and/or for stimulating epidermal renewal and therefore for combating intrinsic and/or extrinsic skin aging.

Thus, the present invention features administration, to an individual subject in need of such treatment, at least one extract of at least one plant of the species Sophora japonica, or composition comprised thereof, and for such period of time as required to elicit the desired response, to promote desquamation of the skin and/or to stimulate epidermal renewal and, therefore, to combat intrinsic and/or extrinsic skin aging.

The species Sophora japonica belongs to the plant genus Sophora which itself belongs to the family Leguminosae. Commonly termed pagoda tree, it is native to central and northern China.

The subject extract may of course be prepared from at least any one of the numerous varieties associated with each of the plant species belonging to the genus Sophora. It will therefore be appreciated why the term Sophora japonica is intended to designate any of the numerous plant varieties associated with each of the plant species belonging to the genus Sophora and particularly to designate the species Sophora japonica.

The extract of a plant of the species Sophora japonica may be any extract prepared from any plant material derived from at least one plant of the genus Sophora.

Thus, the plant of the species Sophora japonica according to the invention may be obtained from plant material derived from the whole plant, or from plant portions such as the leaves, the stems, the flowers, the petals, the seeds, the roots or dedifferentiated cells.

By the expression “dedifferentiated plant cells” is intended any plant cell which does not exhibit any of the characters of a particular specialization and which is capable of living or survival by itself and not in dependence on other cells.

Preferably according to the invention, an extract prepared from green leaves is used.

The extract of at least one plant of the species Sophora japonica may be any extract prepared from any plant material derived from at least one plant of the species Sophora japonica cultured in vivo or derived from in vitro culture.

By the expression “in vivo culture” is intended to mean any culture of the conventional type, namely, in soil, in the open air, or in a greenhouse, or, alternatively, soil-free.

By the expression “in vitro culture” are intended all of the techniques known to this art which make it possible to artificially obtain a plant or a portion of a plant. The selection pressure imposed by the physicochemical conditions during the growth of the plant cells in vitro makes it possible to obtain a standard plant material which is available throughout the year, unlike the plants cultured in vivo.

Preferably according to the invention, a plant derived from in vivo culture is used.

Any technique of extraction known to this art is suitable to prepare the extract contained in the compositions according to the invention.

Particularly exemplary are aqueous or alcoholic extracts, or extracts using an organic solvent.

By the expression “aqueous solvent” is intended any solvent comprising, totally or in part, water. Thus exemplary are water itself, aqueous/alcoholic solvents in any proportion, or, alternatively, solvents comprising water and a compound such as propylene glycol, in any proportion.

Among the alcoholic solvents, ethanol is particularly representative.

An extract prepared by the method described in French patent application No. 95-02379, assigned to the assignee hereof, is also suitable.

Thus, in a first stage, the plant material is ground in an aqueous solution in a cold state, in a second stage, the particles in suspension are removed from the aqueous solution derived from the first stage, and in a third stage, the aqueous solution derived from the second stage is sterilized.

This aqueous solution corresponds to the extract.

On the other hand, the first stage may be advantageously replaced by a simple operation of freezing the plant tissues (for example at −20° C.), followed by an aqueous extraction comprising the second and third stages described above.

Regardless of the technique of preparation according to the invention, the subsequent stages intended to promote preservation and/or stabilization may be included without thereby modifying the actual nature of the extract. Thus, for example, the extract obtained may be freeze-dried by any conventional freeze-drying methods. A powder is thus obtained which may be used directly or mixed in an appropriate solvent prior to use.

Commercial extracts, such as the dry extract of a plant of the species Sophora japonica marketed by Solabia under the trademark Sophorine®, are preferred extracts according to the invention.

This invention also features cosmetic/dermatological compositions for promoting desquamation of the skin and/or for stimulating epidermal renewal and, therefore, for combating intrinsic and/or extrinsic skin aging comprising at least one extract of at least one plant of the species Sophora japonica, formulated into appropriate vehicle, diluent or carrier thereof.

The amounts of extract of at least one plant of the species Sophora japonica which is suitable according to the invention quite obviously depends on the desired effect and should be an amount which is effective for promoting desquamation of the skin and/or for stimulating epidermal renewal and therefore for combating intrinsic and/or extrinsic skin aging.

For example, the quantity of extract of at least one plant of the species Sophora japonica advantageously administered according to the invention may range from 0.001% to 20% by weight, and preferably from 0.01% to 10% of the total weight of the composition.

Too, the present invention features a nontherapeutic regime or regimen for promoting desquamation of the skin and/or for stimulating epidermal renewal and therefore for combating intrinsic and/or extrinsic skin aging, comprising topically applying a cosmetic composition which comprises at least one extract of at least one plant of the species Sophora japonica onto the skin.

The subject compositions may be provided in any of the known galenic forms, such as, for example, in the form of an emulsion, in particular an oil-in-water or water-in-oil emulsion, or even in the form of a multiple emulsion.

Same may also be provided in the form of an optionally gelled, aqueous solution, or in the form of a lotion, for example a two-phase solution, a cream, an ointment, a milk, or even a mousse.

The compositions of the invention may be ingested, injected or topically applied onto the skin (over any cutaneous region of the body), the hair, the nails or the mucous membranes (buccal, jugal, gingeval, genital, conjunctival). According to the particular mode of administration, the compositions according to the invention may be provided in all of the galenic forms normally employed.

For topical application onto the skin, the composition may be in the form, in particular, of an aqueous or oily solution, or of a dispersion of the lotion or serum type, of emulsions having a liquid or semiliquid consistency of the milk type, which are obtained by dispersing a fatty phase in an aqueous phase (O/W) or conversely (W/O), or of suspensions or emulsions having a soft consistency of the aqueous or anhydrous gel or cream type, or, alternatively, of microcapsules or microparticles, or of vesicular dispersions of the ionic and/or nonionic type. These compositions are formulated according to the usual techniques.

They may also be applied to the hair in the form of aqueous, alcoholic or aqueous/alcoholic solutions, or in the form of creams, gels, emulsions, mousses, or, alternatively, in the form of aerosol compositions also comprising a pressurized propellant.

The compositions according to the invention may also be compositions for hair care, and in particular a shampoo, hair-setting lotion, a treatment lotion, a hair-styling cream or gel, a dyeing (in particular oxidation dyeing) composition optionally in the form of dyeing shampoos, restructuring lotions for the hair, a permanent waving composition (in particular a composition for the first stage of a permanent waving), a lotion or gel against hair loss, an antiparasitic shampoo, and the like.

For injection, the compositions are advantageously provided in the form of an aqueous or oily lotion, or in the form of a serum. For the eyes, same may be provided in the form of drops and for ingestion, in the form of capsules, granules, syrups or tablets.

The amounts of the various constituents of the compositions according to the invention are those conventionally used in the fields under consideration.

The compositions according to the invention may also be solid preparations constituting cleansing cakes or soaps.

The compositions may also be packaged in the form of an aerosol composition, also comprising a pressurized propellant.

When the composition is an emulsion, the proportion of the fatty phase may range from 5% to 80% by weight, and preferably from 5% to 50% by weight relative to the total weight of the composition. The oils, waxes, emulsifiers and coemulsifiers included in the composition in the form of an emulsion are selected from among those conventional to the cosmetic field. The emulsifier and coemulsifier are advantageously present in the composition in a proportion ranging from 0.3% to 30% by weight, and preferably from 0.5 to 20% by weight relative to the total weight of the composition. The emulsion may, in addition, contain lipid vesicles.

When the composition is an oily gel or solution, the fatty phase may constitute more than 90% of the total weight of the composition.

The compositions of the invention are suited for cosmetic or pharmaceutical applications. Preferably, the compositions of the invention are compositions for cosmetic applications.

In known fashion, the cosmetic compositions may also contain the usual additives and adjuvants in the cosmetic field, such as hydrophilic or lipophilic gelling agents, hydrophilic or lipophilic active agents, preservatives, antioxidants, solvents, perfumes, fillers, screening agents, odor absorbers and colorants. The amounts of these various additives and adjuvants are those conventionally formulated in the cosmetic field, and range, for example, from 0.01% to 10% of the total weight of the composition. These additives and adjuvants, depending on their nature, may be formulated into the fatty phase, into the aqueous phase and/or into the lipid spherules.

Exemplary oils or waxes which may be included according to this invention are the mineral oils (petroleum jelly), vegetable oils (liquid fraction of shea butter, sunflower oil), animal oils (perhydrosqualene), synthetic oils (Purcellin oil), silicone oils or waxes (cyclomethicone) and fluorinated oils (perfluoropolyethers), beeswaxes, carnauba or paraffin waxes. It is also possible to add to these oils fatty alcohols and fatty acids (stearic acid).

Exemplary emulsifiers include glyceryl stearate, polysorbate 60 and the PEG-6/PEG-32/Glycol Stearate mixture marketed under the trademark Tefose® 63 by Gattefosse.

Exemplary solvents include the lower alcohols, in particular ethanol and isopropanol, propylene glycol.

Exemplary hydrophilic gelling agents include carboxyvinyl polymers (carbomer), acrylic copolymers such as acrylate/alkyl acrylate copolymers, polyacrylamides, polysaccharides such as hydroxypropyl cellulose, natural gums and clays, and exemplary lipophilic gelling agents include modified clays such as bentones, metal salts of fatty acids such as aluminum stearates and hydrophobic silica, ethyl cellulose, polyethylene.

The subject compositions may contain other hydrophilic active agents such as proteins or protein hydrolysates, amino acids, polyols, urea, allantoin, sugars and sugar derivatives, water-soluble vitamins, plant extracts and hydroxy acids.

And exemplary lipophilic active agents include retinol (vitamin A) and derivatives thereof, tocopherol (vitamin E) and derivatives thereof, essential fatty acids, ceramides, essential oils, salicylic acid and derivatives thereof.

According to the invention, the subject compositions may combine at least one extract with other active agents. Exemplary such active agents include:

(a) agents enhancing the regrowth and/or reducing hair loss, and which have already been described for this activity, such as, for example, nicotinic acid esters, including, in particular, tocopheryl nicotinate, benzyl nicotinate and C₁-C₆ alkykl nicotinates such as methyl or hexyl nicotinates, pyrimidine derivatives, such as 2,4-diamino-6-piperidinopyrimidine 3-oxide or “Minoxidil” described in U.S. Pat. No. 4,139,619 and 4,596,812, the agents promoting hair regrowth such as those described in the European patent application published under the number 0648488, assigned to the assignee hereof;

(b) agents modulating skin differentiation and/or proliferation and/or pigmentation, such as retinoic acid and its isomers, retinol and its esters, vitamin D and derivatives thereof, estrogens such as estradiol, kojic acid or hydroquinone;

(c) antibacterials such as clindamycin phosphate, erythromycin or antibiotics of the tetracycline class;

(d) antiparasitic agents, in particular metronidazole, crotamiton or pyrethrinoids;

(e) antifungal agents, in particular the compounds belonging to the imidazole class, such as econazole, ketoconazole or miconazole or their salts, polyene compounds such as amphotericin B, compounds of the allylamine family such as terbinafine, or octopirox;

(f) antiviral agents such as acyclovir;

(g) steroidal anti-inflammatory agents such as hydrocortisone, betamethasone valerate or clobetasol propionate, or nonsteroidal anti-inflammatory agents such as, for example, ibuprofen and its salts, diclofenac and its salts, acetyl salicylic acid, acetaminophen or glycyrrhizic acid;

(h) anaesthetic agents such as lidocaine hydrochloride and its derivatives;

(i) antipruritic agents such as thenaldine, trimeprazine or cyproheptadine;

(j) keratolytic agents such as α- and β-hydroxycarboxylic or β-ketocarboxylic acids, their salts, amides or esters and more particularly hydroxy acids such as glycolic acid, lactic acid, salicylic acid, citric acid and, in general, fruit acids, and 5-n-octanylsalicylic acid;

(k) anti-free radical agents such as a α-tocopherol or its esters, superoxide dismutases, certain metal chelators or ascorbic acid and its esters;

(l) antiseborrhoeic agents such as progesterone;

(m) antidandruff agents such as octopirox or zinc pyrithione;

(n) anti-acne agents such as retinoic acid or benzoyl peroxide;

(o) extracts of plant, marine or bacterial origin.

Other compounds may also be included in the above list, namely, for example, Diazoxide, Spiroxazone, phospholipids such as lecithin, linoleic and linolenic acids, salicylic acid and derivatives thereof which are described in FR-2,581,542, such as the salicylic acid derivatives bearing an alkanoyl substituent having from 2 to 12 carbon atoms at the 5-position of the benzene ring, hydroxycarboxylic or ketocarboxylic acids and their salts, lactones and their corresponding salts, anthralin, carotenoids, eicosatetraenoic and eicosatrienoic acids and their esters and amides, vitamin D and derivatives thereof, extracts of plant or bacterial origin.

Thus, in one particular embodiment, the compositions according to the invention also comprise at least one active agent selected from among antibacterial agents, antiparasitic agents, antifungals, antivirals, anti-inflammatory agents, antipruritic agents, anaesthetics, keratolytic agents, anti-free radical agents, antiseborrhoeic agents, antidandruff agents, anti-acne agents and/or agents reducing skin differentiation and/or proliferation and/or pigmentation, extracts of plant, marine or bacterial origin.

The pharmaceutical compositions according to the invention are conveniently administered via the parenteral or enteral route, or, alternatively, via the topical route. Preferably, the pharmaceutical compositions are administered by topical application onto the skin.

In order to further illustrate the present invention and the advantages thereof, the following specific examples are given, it being understood that same are intended only as illustrative and in nowise limitative.

### EXAMPLE 1

### Activity of a Sophora japonica Extract in the Cell Detachment Model in Vitro in Humans

Plant extract: Dry extract of a plant of the species Sophora japonica marketed by Solabia under the trademark Sophorine®

Skin and set-up: The skin used was obtained from an abdominal plastic surgery operation. The skin was placed flat on a glass plate, covered with a bottomless 96-well plate, and then clamped so as to delimit 96 leaktight wells. To avoid drying, the entire experiment was carried out with the skin immersed in a phosphate-buffered saline (PBS) bath.

The product was prepared by diluting in PBS in order to obtain the concentrations: 1%, 0.5% and 0.1%.

Fifty microliters of each dilution of the product were distributed over the skin in the leaktight wells; the incubation was carried out for 18 hours at 20° C. The control wells received 50 microliters of PBS. The treatments were carried out in triplicate. After a gentle and reproducible pipetting, the content of the wells was removed and placed in another plate. The wells were washed and the solutions were combined with the 50 microliters collected.

Visualization and Relative Quantification of the Corneocytes Released

After dilution, a fraction (20·μl) of each sample (containing the possible corneocytes) was transferred onto nitrocellulose (Hybond ECL, amersham), using a “Slot Blot” matrix (Milliblot, Millipore) allowing subsequent densitometric quantification. The membrane was then saturated overnight, at 4° C., in PBS/0.05% Tween (PBST) containing 1% skimmed milk.

After washing in PBST, the structures derived from the corneocytes were labeled with an anti-total corneocyte polyclonal antibody (1 h, 20° C.) and then with a peroxidase-anti-rabbit immunoglobulin antibody conjugate (Tebu; 1 h, 20° C.). The bound peroxidase was visualized by chemiluminescence (enhance chemiluminescence, ECL, Amersham) on Kodak MP film.

The image acquisition was performed on Gel print 2000i (Biophotonics Corp.). The densitometric analyses were obtained using the One-D-Scan software (Scanalytics).

Data processing: The raw data were transferred and processed under the PRISM® software (Graph Pad Software). The intergroup comparisons were carried out by variance analysis (ANOVA) using DUNNETT's multiple comparison test.

The results obtained are reported in the following Table:

TABLE

| | | Acacia | | | |
|---|---|---|---|---|---|
| | Control | honey: 5% | E: 0.1% | E: 0.5% | E: 1% |
| Activity in | 100 | 427 | 254 | 246 | 409 |
| % | | p < 0.01 | p < 0.05 | p < 0.05 | p < 0.01 |

E: dry extract of a plant of the species Sophora japonica marketed by Solabia under the trademark Sophorine®.

The activity of the Sophora japonica extract on cell detachment was marked; it was equivalent to that of the reference, acacia honey.

In conclusion, the results presented above indeed confirmed the role of the Sophora japonica extract in regulating the detachment of differentiated keratinocytes.

### EXAMPLES 2

The following Examples 2-6 are of specific compositions according to the present invention, formulated as indicated. These compositions were formulated by simply intimately admixing the several constituents thereof.

### EXAMPLE 2

Composition 1—Face Milk

Composition 1 - Face milk:

| | |
|---|---|
| Sophorine ® | 1.0% |
| 3-[(2-Methoxy)ethoxymethoxy]propyl | 1.0% |
| 1,2-di-(10-hydroxydec-2-enoate) | |
| Glyceryl monostearate, polyethylene glycol | 3.0% |
| stearate (100 EO) | |
| Carboxyvinyl polymer | 0.4% |
| Stearyl alcohol | 0.7% |
| Soyabean proteins | 3.0% |
| NaOH | 0.4% |
| Preservative qs | |
| Water qs | 100% |

This composition was provided in the form of a face milk which had good cosmetic properties and which was smooth and comfortable on application.

The pH of the composition was about 5.5.

### EXAMPLE 3

Composition 2—Lotion

Composition 2 - Lotion:

| | |
|---|---|
| Sophorine ® | 0.5% |
| 2-Ethylhexyl palmitate | 10.0% |
| Cyclopentadimethylsiloxane | 20.0% |
| Butylene glycol | 5.0% |
| Preservative qs | |
| Water qs | 100% |

This lotion, which does not contain a surfactant, promoted desquamation of the skin.

### EXAMPLE 4

Composition 3—Milk

Composition 3 - Milk:

| | |
|---|---|
| Octyl palmitate | 35.0% |
| Glycerine | 2.0% |
| Sophorine ® | 1.5% |
| Crosslinked acrylate/C₁₀-C₃₀ | 0.1% |
| alkyl acrylate polymer | |
| Triethanolamine | 0.1% |
| Wheat amino acids | 1.0% |
| Preservative qs | |
| Water qs | 100% |

The milk obtained, which contained no surfactant, had good cosmetic properties.

### EXAMPLE 5

Composition 4—Face Gel

Composition 4 - Face gel:

| | |
|---|---|
| Glycerine | 10.00% |
| Sophorine ® | 0.05% |
| Disodium cocoamphodiacetate | 1.00% |
| Preservative qs | |
| Water qs | 100% |

The gel obtained possessed good cosmetic properties.

### EXAMPLE 6

Composition 5—Water-based Cleansing Gel

Composition 5 - Water-based cleansing gel:

| | |
|---|---|
| Butylene glycol | 7.0% |
| Sodium lauroyl sarcosinate | 4.0% |
| Sophorine ® | 0.1% |
| Triethanolamine | 0.8% |
| Carbomer | 0.5% |
| Preservative qs | |
| Water qs | 100% |

The gel obtained possessed good cosmetic properties.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime/regimen for promoting desquamation of the skin of an individual subject in need of such treatment, comprising administering to such individual, for such period of time as required to elicit the desired response, a thus effective amount of at least one extract of at least one plant of the species Sophora japonica.

2. A regime/regimen for stimulating the epidermal renewal of the skin of an individual subject in need of such treatment, comprising administering to such individual, for such period of time as required to elicit the desired response, a thus effective amount of at least one extract of at least one plant of the species Sophora japonica.

3. A regime/regimen for combating intrinsic and/or extrinsic aging of the skin of an individual subject in need of such treatment, comprising administering to such individual, for such period of time as required to elicit the desired response, a thus effective amount of at least one extract of at least one plant of the species Sophora japonica.

4. The regime/regimen as defined by any of claims 1 to 3, said at least one extract having been obtained from at least one plant of the species Sophora japonica cultivated in vitro.

5. The regime/regimen as defined by any of claims 1 to 3, said at least one extract having been obtained from at least one plant of the species Sophora japonica cultivated in vivo.

6. The regime/regimen as defined by any of claims 1 to 3, comprising topically applying said at least one extract of at least one plant of the species Sophora japonica onto the skin of such individual subject in need of such treatment.

7. The regime/regimen as defined by any of claims 1 to 3, said at least one extract of at least one plant of the species Sophora japonica having been obtained from the leaves, stems, flowers, petals, seeds, roots, and/or dedifferentiated cells thereof.

8. A cosmetic/dermatological composition for promoting desquamation and/or for stimulating epidermal renewal and/or for combating intrinsic/extrinsic aging of the skin, comprising from 0.001% to 20% by weight of at least one extract of at least one plant of the species Sophora japonica, formulated into a cosmetically/dermatologically acceptable vehicle, diluent or carrier therefor.

9. The cosmetic/dermatological composition as defined by claim 8, comprising from 0.01% to 10% by weight of said at least one extract of at least one plant of the species Sophora japonica.

10. The cosmetic/dermatological composition as defined by claim 8, formulated as an emulsion, cream, gel, lotion, milk, ointment, mousse, a solid, syrup or aerosol.

11. The cosmetic/dermatological composition as defined by claim 8, formulated as tablets, capsules, or granules.

12. The cosmetic/dermatological composition as defined by claim 8, formulated for hair care.

13. The cosmetic/dermatological composition as defined by claim 8, further comprising an effective amount of at least one active agent selected from the group consisting of an antibacterial agent, antiparasitic agent, antifungal, antiviral, anti-inflammatory agent, antipruritic agent, anaesthetic, keratolytic agent, anti-free radical agent, antiseborrhoeic agent, antidandruff agent, anti-acne agent and/or agent reducing skin differentiation and/or proliferation and/or pigmentation, extract of plant, marine or bacterial origin, or combination thereof.

14. A topically applicable cosmetic/dermatological composition for promoting desquamation and/or for stimulating epidermal renewal and/or for combating intrinsic/extrinsic aging of the skin, comprising from 0.001% to 20% by weight of at least one extract of at least one plant of the species Sophora japonica, formulated into a topically applicable, cosmetically/dermatologically acceptable vehicle, diluent or carrier therefor.

15. The topically applicable cosmetic/dermatological composition as defined by claim 14, further comprising an effective amount of at least one active agent selected from the group consisting of an antibacterial agent, antiparasitic agent, antifungal, antiviral, anti-inflammatory agent, antipruritic agent, anaesthetic, keratolytic agent, anti-free radical agent, antiseborrhoeic agent, antidandruff agent, anti-acne agent and/or agent reducing skin differentiation and/or proliferation and/or pigmentation, extract of plant, marine or bacterial origin, or combination thereof.
